(19) United States
(12) Patent Application Publication
(10) Pub. No.: US 2020/0205838 A1
(43) Pub. Date: Jul. 2, 2020

(54) Method for Transfemoral Percutaneous Establishment of Retrograde Blood Flow

(71) Applicant: Daniel E. Walzman, Bergenfield, NJ (US)

(72) Inventor: Daniel E. Walzman, Bergenfield, NJ (US)

(21) Appl. No.: 16/350,729

(22) Filed: Dec. 26, 2018

(51) Int. Cl. A61B 17/12; A61B 17/3207; A61M 25/10

(52) U.S. Cl. CPC A61B 17/12109; A61B 17/1204; A61B 8/06; A61M 25/104; A61B 17/3207

(57) ABSTRACT

A method for transfemoral percutaneous establishment of retrograde blood flow. More particularly, a transfemoral percutaneous approach to an interventional procedure on the neurovasculature performed through a transfemoral access while retrograde blood flow is established from the internal vessel artery to a second vessel.

### CROSS-REFERENCES

This application claims the benefit of priority to provisional application Ser. No. 62/707,588 filed Nov. 8, 2017 (8 Nov. 2017), subject to revival pursuant to 35 U.S.C. 119(e)(3) and 37 C.F.R § 1.7(b).

### FEDERALLY FUNDED R&D

None

### BACKGROUND OF THE INVENTION

### Field of the Invention

This invention relates in minimally invasive interventional, medical procedures to revascularize stenosed or thrombosed veins and arteries. More particularly, the present invention pertains to the application of percutaneous approaches to establishing retrograde blood flow during medical procedures.

### Background Art

Vascular atherosclerotic disease usually consists of deposits of plaque, narrowing junctions between a common vessel and an internal vessel.

These deposits increase the risk of embolic matter being generated and entering the cerebral vasculature, leading to neurologic consequences such as transient ischemic attacks or strokes.

Several therapies are employed for treating atherosclerotic and stenotic carotid artery disease. The first is open surgical endarterectomy; the second is percutaneous endovascular angioplasty and stenting.

Both approaches can result in embolic release into the cerebral vasculature. Said release typically results in injury or death.

Some prior art uses distal filters, but the filtration is incomplete. Furthermore, in order to place the filter, the lesion must first be crossed without said embolic protection. During this maneuver debris can embolize to the vascular bed of healthy tissue and cause ischemic injury.

Another protective procedure is the use of balloons placed proximally to medical intervention sites which block flow, and thereby prevent embolic debris from causing damage. While stopping flow can be very effective, some prior methods were difficult to perform and, hence, had limited use.

A significant improvement for prevention of embolic difficulties is the reversal of blood flow using a pump and filter. This avoids difficulties arising from embolic debris. This method for treatment of carotid artery disease is currently introduced via means other than transfemoral percutaneous procedures.

The transradial approach (TRA) was introduced, for angiography and was later applied for percutaneous coronary intervention (PCI). The radial artery has proven to be a challenging but safe route towards the coronary arteries and is the preferred route for percutaneous coronary procedures in electively treated patients. However, the transfemoral approach (TFA) is normally use for primary PCI, mostly in fear of longer door-to-balloon times and worse procedural outcomes.

Existing methods for executing direct access to the carotid circulation by cut down for endovascular neuro-interventions are generally initiated through a small transverse neck incision just above the clavicle. The common carotid artery (CCA) is carefully dissected free circumferentially. After obtaining vascular control with vessel loupes, a purse-string suture is placed in the CCA. Puncture of the artery in the center of the purse string is followed by navigation of a wire, and then a sheath, into the CCA. The neuro-intervention is then carried out. At the conclusion of the procedure, the sheath is removed from the CCA and the purse string tied to secure the artery.

In some prior art, an incision is made proximal to and above the clavicle to expose the common carotid artery. A flexible sheath is placed directly into the carotid artery and connected to a system that is capable of reversing the flow of blood away from the brain to protect against fragments of plaque that may come loose during the procedure. The blood is filtered and returned through a second sheath placed in the femoral vein in the patient's thigh. This system allows balloon angioplasty and stenting to be performed while blood flow is reversed. After the stent is placed successfully to stabilize the plaque in the carotid artery, flow reversal is turned off and blood flow to the brain resumes in its normal direction. To treat carotid artery stenosis (narrowing), the current procedure devised by Silk Roads tries to combine the best parts of stenting while avoiding the most high-risk and difficult parts of standard open carotid endarterctomy.

The current procedures exist to establish retrograde (reversal) of blood flow in vessels. Said procedures have been taught exclusively for arterial flow.

It is generally understood in the medical community that retrograde of blood flow could theoretically be used in a vein, but the establishment of retrograde of blood flow for vessels other than arteries is without value because the kind of athrosclerotic plaque and narrowing in a symptomatic way is pretty much unheard of in veins and the establishment of retrograde of blood flow was designed and used exclusively to ameliorate or eliminate medical procedure difficulties associated with said arthrosclerotic plaque and said narrowing or arterial thrombi.

The present invention teaches a method that may be effective for the treatment of deep vein thrombosis. In short, prior art teaches away from the use of establishment of retrograde of blood flow for veins, because such a system would often result in massive and potentially life-threatening blood loss. The current invention applies a combination of known elements but it is not likely to be obvious because it yields unpredictable results, namely the amelioration or elimination of medical procedure difficulties associated with said athrosclerotic plaque and said narrowing via percutaneous routes by providing a percutaneous endovascular means to arrest and reverse flow. It also overcomes prior limitations to the possibility of using flow reversal to minimize distal emboli during removal of intravascular thrombus or other intravascular debris in veins and large arteries.

Consider Criado (U.S. Pat. No. 9,789,242 Oct. 17, 2017) entitled “Methods and systems for establishing retrograde carotid arterial blood flow” which requires an open surgical cut-down on the Common Carotid Artery (CCA) above the clavicle. A custom short sheath is then place into the carotid artery. Criado teaches that the sheath is attached to a filter pump system, and the other side of their pump is attached to another sheath that is inserted in the femoral vein. After both sheaths are in place and the circuit is attached, a clamp or tourniquet is placed on the carotid artery either just proximal to the sheath (below it) or around the sheath, to arrest normal antegrade flow. The pump is then turned on, which reverses flow in the carotid artery. The blood flows out into the circuit, through the filter, and back into the patient through the femoral vein sheath, for a net of minimal blood loss. Under x-ray guidance a wire is then passed across the plaque further up in the carotid artery, then a balloon is advanced over the wire and dilated, to widen the narrowing in the artery at the plaque. The balloon is deflated and removed over the wire, which is left in place. A stent is then advanced over the wire and deployed across the narrowing. After removing the stent delivery system additional angiographic images are done. Additional balloon angioplasties can then optionally be done as needed. Then the wire is removed. The short carotid sheath is removed and the artery is repaired by sutures (ie tying the purse-string suture) and the wound above this is then closed.

Additionally, the femoral vein sheath is removed and hemostasis is achieved with a percutaneous closure device or with external manual compression. The reversal of flow in the carotid artery throughout the wire crossing, angioplasty and stenting prevents and debris that might break off from traveling to the brain and causing embolic strokes. Instead, the flow is reversed, all the emboli go out into the circuit, is filtered, and returned to the femoral vein.

The present invention teaches a method to replace the open surgical cut-down and placement of a custom short carotid sheath with a purely percutaneous approach, most often transfemorally. Via standard transfemoral approaches, a balloon guide catheter (BGC) such as the Stryker Flowgate (https://www.strykerneurovascular.com/products/ais/flowgate-balloon-guide-catheter) or Medtronic Cello can be introduced into the common carotid artery under fluoroscopic guidance, using standard interventional/angiographic techniques. In select difficult arch anatomy cases, various difficult angle access catheters previously described by Walzman (patents pending) can optionally be used to help position the BGC. The transfemorally placed balloon guide can then be used instead of the custom short sheath and clamp/tourniquet. Inflating the balloon on the BGC has the same flow arrest properties as the clamp/tourniquet in standard open technique, and the Silk Road circuit or a similar filter pump system can be attached to the t proximal end of the BGC instead of the standard practice of attaching the filter pump system to the proximal end of the custom short sheath. The balloon guide catheter sheath is attached to the filter pump system, and the other side of the pump is attached to another sheath that was inserted in the femoral vein. After both sheaths are in place and the circuit is attached, the balloon on the BGC is inflated, to arrest normal ante-grade flow. The pump is then turned on, which reverses flow in the carotid artery. The blood flows out into the circuit, through the filter, and back into the patient through the femoral vein sheath, for a net of minimal blood loss. The circuit can provide a high rate of flow that will overcome any potential collateral backflow pressure from the external carotid as well, to ensure flow in the internal carotid artery is effectively reversed. Under x-ray guidance a wire is then passed through the balloon guide catheter, typically but not exclusively via the transfemoral approach, across the plaque further up in the carotid artery. Then an angioplasty balloon is advanced over the wire and dilated, to widen the narrowing in the artery at the plaque. The angioplasty balloon is deflated and removed over the wire, which is left in place. A stent is then advanced over the wire and deployed across the narrowing. After removing the stent delivery system additional angiographic images are done. Additional balloon angioplasties can then optionally be done as needed. Then the wire is removed. At the end of the procedure the balloon is deflated and the pump is turned off. The BGC is then removed. Femoral hemostasis can be achieved manually or with a standard closure device (such as Angioseal).

The femoral vein sheath is removed and hemostasis is achieved with a percutaneous closure device (such as Mynx, off label) or manually.

During various stages of the procedure, such as the wire crossing, angioplasty, and stenting, debris can break off and enter the blood. Reversing the blood flow in the Internal carotid artery prevents such debris that might break off from traveling to the brain and causing embolic strokes. Instead the flow is reversed, the blood with all the emboli go out into the circuit, is filtered, and the blood returned to the femoral vein. In my innovative technique above the BGC replaces the custom short sheath in the common carotid artery and the clamp or tourniquet used with it. Inflating the balloon on the BGC arrests flow in the carotid, and attaching and activating their filter pump reverses flow.

The prior art has described similar ways to use a type of BGC to reverse flow in the carotid during stenting, but have never taught to do so with proprietary pump and filter.

The Silk Road proprietary pump filter system may apply higher flow rates than prior art versions, which more often relied on passive flow. This can provide more optimal embolic protection. Furthermore, prior art teaches of using passive flow reversal, which necessitates placing an additional balloon in the external carotid artery to occlude that vessel as well and prevent backflow, thus further complicating the procedure.

Similar flow reversal techniques can also be used during endovascular and/or open arterial thrombectomy procedures. The reversal of flow optimizes removal of clot, and minimizes potential for distal emboli and their attendant ischemic risks. Additional, optionally simultaneous, optional irrigation and/or maceration may also be used to further enhance clot removal.

The present invention also teach a method for the establishment of retrograde blood flow in veins which ameliorates or eliminates the medical difficulty associated with the establishment of retrograde blood flow in veins. Prior art teaches the use of an aspiration system such as Penumbra's or Angio Dynamics Angiovac to establish retrograde blood flow in veins, however said system has the potential for massive blood loss when used for large veins. They also don't utilize a balloon to arrest flow. Furthermore, they offer no mechanism to limit potentially life-threatening blood loss without utilizing a cardiopulmonary type temporary bypass circuit, with its attendant costs, risks, and potential time delays for team assembly. Additionally, most hospitals do not have cardiac bypass capabilities, and cannot offer adjunctive use of a cardiopulmonary type temporary bypass circuit. Thus, the current invention, which ameliorates the potential for massive blood loss when used, can also be easily accomplished in any facility, and without an additional specialty support team of experts. Thus, the current invention surmounts the medical difficulties associated with the prior art's standard procedure for the establishment of retrograde blood flow in veins.

As described, the prior art also teaches the use of the employment of a bypass machine. This option for the establishment of retrograde blood flow in veins is very complex, expensive (need a dedicated perfusionist team), and adds additional risk. With the present invention that teaches the use of a high flow filter pump or a similar system, that filters the emboli out and returns the aspirated blood to the patient, many of these hurdles can be overcome.

The core concept for use of a vacuum filter system in the treatment of intravenous thrombus such as DVT is to use a balloon guide catheter—from above or below, to occlude the vessel, and then use aspiration alone, aspiration in combination with irrigation or maceration, or all three together, often simultaneously, to remove clot in the vein. When the BGC is placed “below” the clot, the balloon is inflated to arrest flow, and the flow will be reversed by aspiration with or without the addition of irrigation, protecting from downstream emboli. If the BGC is introduced in the opposite direction, “above” the clot, the expected antegrade flow with the aspiration will take the emboli into the BGC, and inflating the balloon will prevent downstream emboli (with or without additional irrigation). This technique can optionally be further supplemented with various rotating irrigating maceration tools previous described by Walzman (Ser. No. 15/258,877) and/or other thrombectomy tools, nonlimiting examples of which include the Argon Cleaner XT and the Boston Scientific Angiojet. All of the blood that is removed from the body via aspiration of the BGC will then be filtered through a pump and filter system, and returned to the patient via a sheath/catheter in another vessel, most often another vein.

Additionally, in select cases where the BGC can be placed above the clot (downstream; non-limiting example—introduced via jugular vein into inferior vena cana, with iliac vein clot). By simultaneously using the a pump vacuum filter circuit, and returning blood via another catheter inserted in a vessel in the patient, more continuous aspiration can be safely used.

The present method also teaches the use of a catheter for aspiration that can have both an attached filter, to capture and/or redirect emboli, as well as a balloon to arrest flow as desired. Additionally, the filter may be semi-permeable and allow blood flow through it, while capturing debris, or non-permeable. In the latter example all blood will be diverted to the aspiration catheter.

Most physicians that employ carotid stents are not trained for carotid cut downs, especially outside the United States. There is a need to allow said physicians to use flow-reversal techniques to minimize embolic risks in carotid stenting, including high flow circuits that maximize safety. The present invention fulfills this need as well.

### SUMMARY OF INVENTION

The present invention discloses a method for transfemoral percutaneous establishment of reverse flow blood circulation in target vessels via incorporating the Silk Road Enroute Neuroprotection filter pump system or a similar closed-circuit filter pump device to ameliorate or prevent the release of emboli into distal vasculature.

The methods are particularly useful for percutaneous endovascular procedures such as arterial angioplasty and stenting as well as thrombectomy of arteries or veins.

### DETAILED DESCRIPTION OF THE DRAWINGS

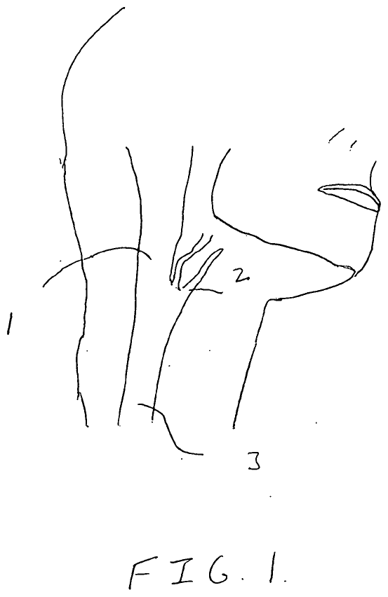

FIG. 1 depicts the vasculature in a patient's neck, including the common target vessel CTV, the internal target vessel ITV, and the external target vessel ETV; labeling ITV as 1, ETV as 2, and CTV as 3.

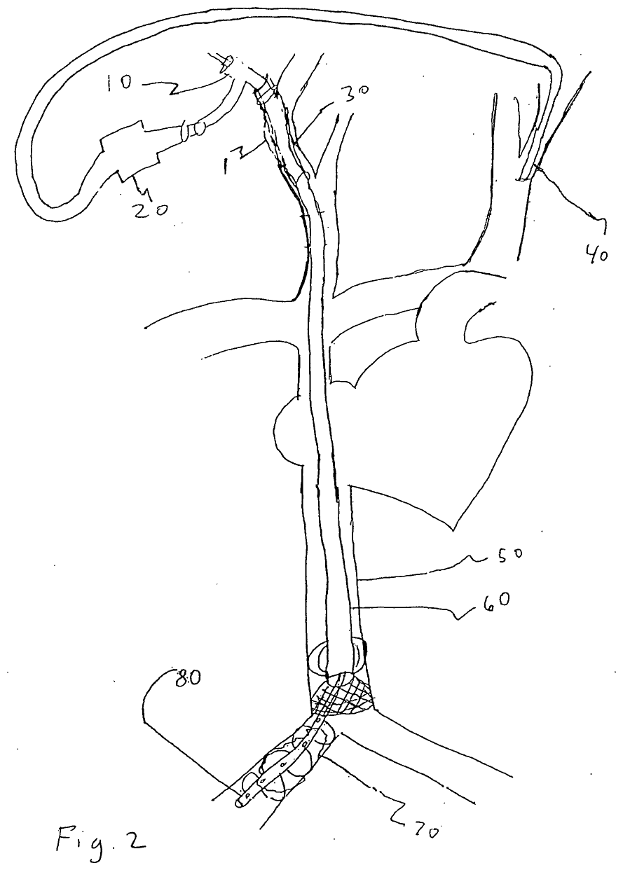

FIG. 2 depicts the hardware's relative positions during the implementation of the present invention's method for transfemoral percutaneous establishment of retrograde blood flow; labeling ITV as 1, RGC as 10, and Silk Road Enroute Neuroprotection circuit as 20, optical sheath as 30, additional sheath in collateral jugular vein for blood return as 40, IVC as 50, filter tip BGC as 60, iliac vein thrombus as 70, rotating, irrigating macerator as 80

### DETAILED DESCRIPTION OF THE INVENTION

The present invention is a method for transfemoral percutaneous establishment of retrograde blood flow during a medical procedure. While all embodiments of said method are designed to implement a single idea (transfemoral percutaneous establishment blood flow), the embodiment may contain a variety of step which depend upon the medical procedure for which they are used.

Angioplasty and/or stenting from proximal approach, with upstream arterial access: Nonlimiting example: Carotid stenosis. Via standard percutaneous approaches (preferentially but optionally transfemoral) a balloon guide catheter (BGC) such as the Stryker Flowgate(https://www.strykerneurovascular.com/products/ais/flowgate-balloon-guide-catheter) or Medtronic Cello can be introduced into the common carotid artery under fluoroscopic guidance, using standard interventional/angiographic techniques. In select difficult arch anatomy cases, various difficult angle access catheters previously described by Walzman (patents pending) can optionally be used to help position the BCG 10. The balloon guide can then be used instead of the Silk Road custom short sheath and clamp/tourniquet described for use in their original procedure. Inflating the balloon on the BCG 10 has the same flow arrest properties as the clamp/tourniquet in standard open technique, and the Silk Road Enroute Neuroprotection circuit 20 or a similar filter pump system can be attached to the proximal end of the BCG 10 (instead of the standard practice of attaching the filter pump system to the proximal end of the custom short sheath). The balloon guide catheter sheath is attached to the filter pump system, and the other side of the pump is attached to another sheath that was inserted in the femoral vein. After both catheters/sheaths are in place and the circuit is attached, the balloon on the BCG 10 is inflated to arrest normal antegrade flow in the carotid artery. The pump is then turned on, which reverses flow in the carotid artery. The blood flows out into the circuit, through the filter, and back into the patient through the femoral vein sheath, for a net of minimal blood loss. Under x-ray guidance a wire is then passed through the balloon guide catheter, typically but not exclusively via the transfemoral approach, across the plaque further up in the carotid artery. Then an angioplasty balloon is advanced over the wire and dilated, to widen the narrowing in the artery at the plaque. The angioplasty balloon is deflated and removed over the wire, which is left in place. A stent is then advanced over the wire and deployed across the narrowing. After removing the stent delivery system additional angiographic images are done. Additional balloon angioplasties can then optionally be done as needed. Then the wire is removed. At the end of the procedure the balloon is deflated and the pump is turned off. The BCG 10 is then removed. Femoral arterial hemostasis can be achieved with manual compression or with a standard closure device (such as Angioseal). The venous sheath can also be removed, and hemostasis can be achieved with manual compression or with a standard closure device (such as Mynx, off-label). Alternatively, for smaller vessels where less blood loss may be anticipated, a blood removal pump such as the Penumbra vacuum system can be optionally used instead of the Enroute Neuroprotection System 20, without a venous sheath and return of removed blood.

Angioplasty and/or stenting with Downstream arterial access: nonlimiting example—axillary artery stenosis—percutaneous transradial or transbrachial access can be obtained in standard fashion, and utilizing standard endovascular techniques a BCG 10 can be advanced into the axillary artery distal to the stenosis. The balloon guide and Enroute Neuroprotection 20 filter pump can then be used instead of typical filter wire._The Silk Road Enroute Neuroprotection 20 circuit or a similar filter pump system can be attached to the proximal end of the BCG 10. The balloon guide catheter sheath is attached to the filter pump system, and the other side of the pump is attached to another sheath 40 that was inserted in the femoral vein (or any large enough vessel). After both catheters/sheaths are in place and the circuit is attached, the balloon on the BCG 10 is inflated to arrest normal antegrade flow in the artery. The pump (Enroute Neuroprotection System 20) is then turned on, which reroutes flow from the artery. The blood flows out into the circuit, through the filter, and back into the patient through the femoral vein sheath, for a net of minimal blood loss. Under x-ray guidance a wire is then passed through the balloon guide catheter across the plaque further proximally in the artery. Then an angioplasty balloon is advanced over the wire and dilated, to widen the narrowing in the artery at the plaque. The angioplasty balloon is deflated and removed over the wire, which is left in place. A stent is then advanced over the wire and deployed across the narrowing. After removing the stent delivery system additional angiographic images are done. Additional balloon angioplasties can then optionally be done as needed. Then the wire is removed. At the end of the procedure the balloon is deflated and the pump is turned off. The BCG 10 is then removed. Arterial hemostasis can be achieved manually or with a standard closure device (off-label). The venous sheath can also be removed, and hemostasis can be achieved with manual compression or with a standard closure device (off-label).

Alternatively, for smaller vessels where less blood loss may be anticipated, a blood removal pump such as the Penumbra vacuum system can be optionally used instead of the Enroute Neuroprotection System 20, without a venous sheath and return of removed blood.)

Arterial thrombectomy from proximal to clot—upstream approach—non-limiting example—mca embolic stroke in distal Right M1 segment: percutaneous transfemoral or other access can be obtained in standard fashion, and utilizing standard endovascular techniques a large long sheath/catheter is placed into the right internal carotid artery. Through that sheath/catheter an appropriately sized BCG 10 can be advanced over a wire (or over a smaller catheter and a wire) into distal ICA or preferentially into the proximal mca, where a sump of blood from unaffected brain territory can be limited, and the potential for emboli to other vascular territories eliminated. A new, more flexible BCG 10 will need to be made for many applications, especially intracranial. The balloon guide catheter and Enroute Neuroprotection 20 filter pump can then be used to reverse flow in the mca. First the balloon is inflated to arrest flow. Then an irrigating and macerating element, such as that described by Walzman elsewhere, or Angiojet, or similar, would be advanced through the thrombus. (Alternatively, a small irrigation catheter alone can be used, without maceration). (Alternatively the simultaneous active irrigation and aspiration can be supplemented with the use of a stent-triever or similar clot retrieval device.) The balloon guide catheter/sheath is attached to the filter pump system, and the other side of the pump is attached to another sheath that was inserted in the femoral vein (or any large enough vessel). After both sheaths are in place and the circuit (Enroute Neuroprotection System 20) is attached. the pump is turned on, which reroutes flow from the artery. This is combined with simultaneous irrigation into and distal to the thrombus, which reverses flow in the mca. Additional simultaneous rotational maceration or jet maceration or similar is also added as needed to break the thrombus into smaller pieces and release its adhesion to the arterial walls. The blood, clots, and irrigant flows out into the circuit, through the filter, and back into the patient through the femoral vein sheath, for a net of minimal blood loss. Injections of contrast through the aspiration catheter or BCG 10 (after adequate back-bleeding) can confirm effective removal of the embolus/thrombus. Then the pump is turned off and the balloon is deflated,and antegrade flow in the mca is restored. Additional angiography can confirm the efficacy of the thrombectomy. The BCG 10 is then removed. The large long carotid sheath/catheter is removed, and arterial hemostasis can be achieved manually or with a standard closure device (off-label). The venous sheath can also be removed, and hemostasis can be achieved with manual compression or with a standard closure device (off-label).

Alternatively, for smaller vessels such as the mca, where acceptable amounts of blood loss may be anticipated, a blood removal pump such as the Penumbra vacuum system can be optionally used instead of the Enroute Neuroprotection System 20, without a venous sheath and return of removed blood.

Arterial Thrombectomy from distal to clot,downstream approach—as above in C, but because the BCG 10 is introduced from downstream to the thrombus/embolus, the flow is never reversed.

Nonlimiting example—ipsilateral percutaneous transfemoral access for an ipsilateral iliac artery thrombus 70: Percutaneous transfemoral access can be obtained in standard fashion, and a BCG 10 can be advanced into the proximal femoral artery or distal iliac artery, distal to the thrombus. The balloon on the BCG 10 is inflated to arrest normal antegrade flow in the artery. An irrigating and macerating element, such as that described by Walzman elsewhere, or Angiojet, or similar, would first be advanced through the thrombus. (Alternatively, a small irrigation catheter alone can be used, without maceration). (Alternatively, the flow circuit created by the vessel occlusion with the balloon, and the simultaneous aspiration and optional active irrigation, can be supplemented with the use of a stent-triever or similar clot retrieval device.) The balloon guide catheter sheath is attached to the filter pump system, and the other side of the pump is attached to another sheath that was inserted in the femoral vein (or any large enough vessel). After both catheters/sheaths are in place and the circuit is attached the pump is then turned on, which reroutes flow from the artery. (Note, in all examples the circuit can alternatively optionally be turned on before crossing the pathological lesion as well). This is combined with simultaneous rotational maceration or jet maceration or similar across the thrombus to break the thrombus into smaller pieces and release its adhesion to the arterial walls. The blood flows out into the circuit (Enroute Neuroprotection System 20), through the filter, and back into the patient through the femoral vein sheath, for a net of minimal blood loss. All thrombi, emboli, and other debris are diverted into the pump circuit and filter, which prevents distal embolic showering and its attendant risks of ischemic injury to tissue. Injections of contrast through the aspiration catheter or other devices can confirm effective removal of the embolus/thrombus. Then the balloon is deflated and the pump is turned off, and antegrade flow in the artery is restored. Additional angiography can confirm the efficacy of the thrombectomy. The BCG 10 is then removed. The arterial sheath hemostasis can be achieved manually or with a standard closure device (off-label). The venous sheath can also be removed, and hemostasis can be achieved with manual compression or with a standard closure device (off-label).

Alternatively, for procedures where acceptable amounts of blood loss may be anticipated, a blood removal pump such as the Penumbra vacuum system can be optionally used instead of the Enroute Neuroprotection System 20, without a venous sheath and return of removed blood.

Alternatively, for procedures where acceptable amounts of blood loss may be anticipated, a blood removal pump such as the Penumbra vacuum system can be optionally used instead of the Enroute Neuroprotection System 20, without a venous sheath and return of removed blood. Venous thrombectomy from downstream only: Nonlimiting example (now referring to FIG. 2 wherein): Iliac clot (or iliofemoral) 70 extending to the junction of the iliac vein with the Inferior Vena cava, with Pertcutaneous Approach from Internal Jugular Vein: Using Standard techniques a large BCG 10 can be advanced into the proximal inferior vena cava (IVC) 50. The balloon guide catheter sheath is attached to the filter pump system (Enroute Neuroprotection System 20), and the other side of the pump is attached to another sheath that was inserted in additional venous sheath in a vein that does not flow downstream to the BGC 10/Filter-tip catheter (ie—contralateral internal jugular vein). After both catheters/sheaths are in place and the circuit is attached, the balloon on the BCG 10 is inflated to arrest normal antegrade flow in the vessel, and/or the filter is deployed (if not yet deployed). The pump is then turned on, which reroutes flow from the vein. Additional simultaneous rotational maceration or jet maceration or similar is also added as needed to break the thrombus into smaller pieces and release its adhesion to the vein's walls. This is combined with simultaneous optional irrigation into and proximal to the thrombus. The blood flows out into the circuit (Enroute Neuroprotection System 20), through the filter, and back into the patient through an additional venous sheath, for a net of minimal blood loss. A macerating element, with optional additional irrigation, such as that described by Walzman elsewhere, or Angiojet, or Argon Cleaner XT or similar is through the thrombus. With flow reversed by aspiration, simultaneous maceration and/or irrigation are added to break up and free up the clot, and promote flow into the BCG 10 and the Enroute Neuroprotection System 20. Emboli to the lungs and/or heart are avoided. Injections of contrast through the BCG 10 or other devices can confirm effective removal of the embolus/thrombus. Then the balloon is deflated and the pump is turned off, and antegrade flow in the vein is restored. Additional angiography can confirm the efficacy of the thrombectomy. The BCG 10 is then removed. Venous hemostasis can be achieved manually or with a standard closure device (off-label). The second venous sheath can also be removed, and hemostasis can be achieved with manual compression or with a standard closure device (off-label).

Alternatively, if using the semipermeable filter tip aspiration catheter, where acceptable amounts of blood loss may be anticipated because only intermittent aspiration can sometimes be used, a blood removal pump such as the Penumbra vacuum system can be optionally used instead of the Enroute Neuroprotection System 20, without a venous sheath and return of removed blood.

Non-limiting example 2: Dialysis Arm AV fistula clot, with Pertcutaneous Approach from Femoral Vein: Using Standard techniques an appropriately sized BCG 10 can be advanced over a wire (or over a smaller catheter and a wire) into axillary vein, brachial vein, or subclavian vein (downstream from the clot). The BGC 10 may optionally have a filter tip 60. For this application the filter can be semipermeable or impermeable. Alternatively, a filter tip aspiration catheter previously described by Walzman (patent pending) alone can be used (without a balloon, since the filter will prevent downstream emboli. Furthermore, since in this example the positioning is downstream, so there is no need for induced flow-reversal). The balloon guide catheter sheath is attached to the filter pump system (Enroute Neuroprotection System 20), and the other side of the pump is attached to another sheath that was inserted in additional venous sheath in a vein that does not flow downstream to the BGC/Filter-tip catheter (ie—internal jugular vein, or contralateral femoral vein). After both catheters/sheaths are in place and the circuit is attached, the balloon on the BCG 10 is inflated to arrest normal antegrade flow in the vessel, and/or the filter is deployed (if not yet deployed). A macerating element, with optional additional irrigation, such as that described by Walzman elsewhere, or Angiojet, or Argon Cleaner, or angioplasty balloon, or similar, would first be advanced through the thrombus. The pump is then turned on, which reroutes flow from the vein. Additional simultaneous rotational maceration or jet maceration or angioplasty or similar is performed as needed to break the thrombus into smaller pieces and release its adhesion to the AV fistula vessel walls. This is combined with simultaneous optional irrigation into and proximal to the thrombus. The blood flows out into the circuit (Enroute Neuroprotection System 20), through the filter, and back into the patient through an additional venous sheath, for a net of minimal blood loss. Emboli to the lungs and/or heart are avoided. Injections of contrast through the aspiration catheter or the BCG 10 or other devicesvcan confirm effective removal of the embolus/thrombus. Then the balloon is deflated and the pump is turned off, and antegrade flow in the vein is restored. Additional angiography can confirm the efficacy of the thrombectomy. The BCG 10 is then removed. Venous hemostasis can be achieved manually or with a standard closure device (off-label). The second venous sheath can also be removed, and hemostasis can be achieved with manual compression or with a standard closure device (off-label). {Alternatively, if using the semipermeable filter tip aspiration catheter, and/or where acceptable amounts of blood loss may be anticipated, sometimes because only intermittent aspiration can sometimes be used, a blood removal pump such as the Penumbra vacuum system can be optionally used instead of the Enroute Neuroprotection System 20, without a venous sheath and return of removed blood. Venous Thrombectomy from upstream only:

Non-limiting example: Iliac clot, with Pertcutaneous Approach from ipsilateral Femoral Vein: Using Standard techniques an appropriately sized BCG 10 can be advanced into the femoral vein or proximal iliac vein (upstream from the clot). The balloon guide catheter sheath is attached to the filter pump system (Enroute Neuroprotection System 20), and the other side of the pump is attached to another sheath that was inserted in additional venous sheath in another vein that does not flow downstream to the BCG 10/Filter-tip 60 catheter (ie—internal jugular vein, or contralateral femoral vein). After both catheters/sheaths are in place and the circuit is attached, the balloon on the BCG 10 is inflated to arrest normal ante-grade flow in the vessel. The pump is then turned on, which reroutes flow from the vessel. A macerating element, with optional additional irrigation, such as that described by Walzman elsewhere, or Angiojet, or Argon Cleaner, or angioplasty balloon, or similar, would be advanced through the thrombus. Then additional simultaneous rotational maceration or jet maceration or angioplasty or similar is performed as needed to break the thrombus into smaller pieces and release its adhesion to the AV fistula vessel walls. This is combined with simultaneous optional irrigation into and proximal and distal to the thrombus. The blood flows out into the circuit (Enroute Neuroprotection System 20), through the filter, and back into the patient through an additional venous sheath, for a net of minimal blood loss. Emboli to the lungs and/or heart are avoided. Injections of contrast through the BCG 10 or other devices can confirm effective removal of the embolus/thrombus. Then the balloon is deflated and the pump is turned off, and ante-grade flow in the vein is restored. Additional angiography can confirm the efficacy of the thrombectomy. The BCG 10 is then removed. Venous hemostasis can be achieved manually or with a standard closure device (off-label). The second venous sheath can also be removed, and hemostasis can be achieved with manual compression or with a standard closure device (off-label). {Alternatively, where acceptable amounts of blood loss may be anticipated, a blood removal pump such as the Penumbra vacuum system can be optionally used instead of the Enroute Neuroprotection System 20, without a venous sheath and return of removed blood.

Venous thrombectomy with combined approach—similar steps to other venous thrombectomy methods described above, optionally using Enroute Neuroprotection System 20 to aspirate, filter and return the blood, but where the macerating element or similar is introduced from the opposite approach. As a non-limiting example—for a femoral vein thrombus—the filter-tip aspiration catheter (or filter tip BGC 60) can be introduced through either IJV into the mid-IVC 50, where the filter is deployed. An additional sheath/catheter 40 is placed in the other IJV for blood return via the Enroute Neuroprotection System 20. The circuit is hooked up. A second catheter/sheath is placed via the popliteal vein ipsilateral to the clot, and through that the irrigating macerator, BS Angiojet, Argon Cleaner, or similar is advanced into and across the clot. Maceration is performed with continuous or intermittent aspiration through the Enroute Neuroprotection System. Intermittent contrast injections can be performed to monitor thrombus removal, with or with put supplemental external ultrasound or IVUS (These can optionally be used in all examples as well). Once adequate clot removal is achieved, the pump is turned off Any balloons are deflated and all devices are removed, with appropriate hemostasis. the semipermeable filter tip aspiration catheter, and/or where acceptable amounts of blood loss may be anticipated, sometimes because only intermittent aspiration can sometimes be used, a blood removal pump such as the Penumbra vacuum system can be optionally used instead of the Enroute Neuroprotection System, without a venous sheath and return of removed blood.

In sum, the present invention also teaches a method using of the Enroute Neuroprotection System; also novel uses of Balloon Guide catheters and B.S. Angiojet, Argon Cleaner, and similar devices (using these protective modalities is the novelty); also novel uses of Penumbra aspiration and similar, as well as

Although the invention has been described in detail in the foregoing embodiments for the purpose of illustration, it is to be understood that such detail is solely for that purpose, and that variations can be made therein by those skilled in the art without departing from the spirit and scope of the invention, except as it may be described by the following claims.

I claim:

1. A method for establishing retrograde blood flow using a transfemoral percutaneous procedure, comprising the steps of:
First, obtaining endovascular access of a common target vessel via percutaneous procedural technique, optionally transfemoral, said access being located at distance of about 2 to 3 inches below a bifurcation location where the patient's common target vessel bifurcates into an internal target vessel and an external target vessel;
Second, positioning a vessel-access sheath through the transfemoral percutaneous procedural incision into the common target vessel, wherein the vessel-access sheath includes an expandable element at some point along its distal 6 inches;
Third, expanding said expandable element so that said expandable element blocks blood flow through the common target vessel past the sheath, and establish a reverse blood flow through the internal target vessel, and into said sheath, wherein blood flows into a shunt of said sheath while the common target vessel remains blocked;
Fourth, actively assisting blood flow from the common target vessel into said sheath and into said shunt.

2. The method of claim 1, further including in said First step, obtaining endovascular access of a common target vessel via transfemoral percutaneous procedural technique, said access being located at distance of about 2 to 3 inches below a bifurcation location where the patient's common target vessel bifurcates into an internal target vessel and an external target vessel.

3. The method of claim 1, further including in said Fourth step filtering any debris from the blood within said shunt; flowing the blood from said shunt to a return location.
